(19) United States
(12) Patent Application Publication
(10) Pub. No.: US 2020/0334998 A1
(43) Pub. Date: Oct. 22, 2020

(54) WEARABLE IMAGE DISPLAY DEVICE FOR SURGERY AND SURGERY INFORMATION REAL-TIME DISPLAY SYSTEM

(71) Applicant: NATIONAL CHENG KUNG UNIVERSITY, Tainan City (TW)

(72) Inventors: Yung-Nien SUN, Tainan City (TW); I-Ming JOU, Tainan City (TW); Chang-Yi CHIU, Tainan City (TW); Bo-Siang TSAI, Tainan City (TW); Yu-Hsiang CHENG, Tainan City (TW); Bo-I CHUANG, Tainan City (TW); Chan-Pang KUOK, Tainan City (TW)

(21) Appl. No.: 16/559,279

(22) Filed: Sep. 3, 2019

(30) Foreign Application Priority Data
Apr. 16, 2019 (TW) 108113269

(51) Int. Cl. G09B 5/02; A61B 90/00; A61B 5/06; G06F 3/14

(52) U.S. Cl. CPC G09B 5/02; A61B 90/37; G09B 23/30; G06F 3/14; A61B 2090/372; A61B 5/064

(57) ABSTRACT

A wearable image display device for surgery includes a display unit, a wireless receiver and a processing core. The wireless receiver wirelessly receives a medical image or a medical instrument information in real-time. The processing core is coupled to the wireless receiver and the display unit for displaying the medical image or the medical instrument information on the display unit.

### CROSS REFERENCE TO RELATED APPLICATIONS

This Non-provisional application claims priority under 35 U.S.C. § 119(a) on Patent Application No(s). 108113269 filed in Taiwan, Republic of China on Apr. 16, 2019, the entire contents of which are hereby incorporated by reference.

### BACKGROUND

### Technology Field

The present disclosure relates to a wearable image display device and system, and in particular, to a wearable image display device for surgery and a surgery information real-time display system.

### Description of Related Art

The operators usually need a lot of trainings for operating a medical instrument before applying to the real patients. In the case of minimally invasive surgery, in addition to operating the scalpel, the operator (e.g. surgeon) also operates the probe of ultrasound image equipment. The allowed error in the minimally invasive surgery is very small, and the operator usually needs a lot of experience to perform the operation smoothly. Thus, the pre-operative training is extraordinarily important. In addition, if the surgeon needs to turn his/her head to look at the image displayed by the medical device during the operation, it is also inconvenient for the operation.

Therefore, it is an important subject to provide a wearable image display device for surgery and a surgery information real-time display system that can assist or train the user to operate the medical instrument.

### SUMMARY

In view of the foregoing, an objective of this disclosure is to provide a wearable image display device for surgery and a surgery information real-time display system that can assist or train the user to operate the medical instrument.

A wearable image display device for surgery comprises a display unit, a wireless receiver, and a processing core. The wireless receiver receives a medical image or a medical instrument information in real-time. The processing core is coupled to the wireless receiver and the display unit for displaying the medical image or the medical instrument information on the display unit.

In one embodiment, the medical image is an artificial medical image of an artificial limb.

In one embodiment, the wearable image display device is a smart glasses or a head mounted display.

In one embodiment, the medical instrument information comprises a location information and an angle information.

In one embodiment, the wireless receiver wirelessly receives a surgery target information in real-time, and the processing core displays the medical image, the medical instrument information or the surgery target information on the display unit.

In one embodiment, the surgery target information comprises a location information and an angle information.

In one embodiment, the wireless receiver wirelessly receives a surgery guidance video in real-time, and the processing core displays the medical image, the medical instrument information or the surgery guidance video on the display unit.

A surgery information real-time display system comprises the above-mentioned wearable image display device for surgery and a server. The server is wirelessly connected with the wireless receiver for wirelessly transmitting the medical image and the medical instrument information in real-time.

In one embodiment, the server transmits the medical image and the medical instrument information through two network sockets, respectively.

In one embodiment, the system further comprises an optical positioning device detecting a position of a medical instrument for generating a positioning signal. The server generates the medical instrument information according to the positioning signal.

As mentioned above, the wearable image display device for surgery and the surgery information real-time display system of this disclosure can assist or train the user to operate the medical instrument. The training system of this disclosure can provide the trainee with a realistic surgical training situation, thereby effectively assisting the trainee to complete the surgical training.

In addition, the surgeon can practice with a simulated surgery on a fake body (e.g. a body phantom) in advance, and then before the actual surgery operation, the surgeon can review the practiced simulated surgery by the wearable image display device for surgery and the surgical information real-time display system for quickly reminding the key points of the operation or the points to be noted.

Moreover, the wearable image display device for surgery and the surgery information real-time display system of this disclosure can be applied to the actual operation process. For example, the medical images (e.g. ultrasonic images) can be transmitted to the wearable image display device for surgery (e.g. a smart glasses), so that the surgeon can view the displayed images without turning head to watch another display screen.

### BRIEF DESCRIPTION OF THE DRAWINGS

The disclosure will become more fully understood from the detailed description and accompanying drawings, which are given for illustration only, and thus are not limitative of the present disclosure, and wherein:

FIG. 1A is a block diagram of a surgery information real-time display system according to an embodiment of this disclosure;

FIG. 1B is a schematic diagram showing that the wearable image display device for surgery of FIG. 1A receives the medical image or the medical instrument information;

FIG. 1C is a schematic diagram showing the transmission between the server and the wearable image display device for surgery of FIG. 1A;

FIG. 1D is a schematic diagram showing that the server of FIG. 1A transmits through two network sockets;

FIG. 2A is a block diagram showing an optical tracking system according to an embodiment of this disclosure;

FIGS. 2B and 2C are schematic diagrams showing the optical tracking system according to an embodiment of this disclosure;

FIG. 2D is a schematic diagram showing a surgical situation 3-D model according to an embodiment of this disclosure;

FIG. 3 is a functional block diagram of a surgery training system according to an embodiment of this disclosure;

FIG. 4 is a block diagram showing a training system for operating a medical instrument according to an embodiment of this disclosure;

FIG. 5A a schematic diagram showing a surgical situation 3-D model according to an embodiment of this disclosure;

FIG. 5B is a schematic diagram showing a physical medical image 3-D model according to an embodiment of this disclosure;

FIG. 5C is a schematic diagram showing an artificial medical image 3-D model according to an embodiment of this disclosure;

FIGS. 6A to 6D are schematic diagrams showing direction vectors of the medical instruments according to an embodiment of this disclosure;

FIGS. 7A to 7D are schematic diagrams showing the training procedure of the training system according to an embodiment of this disclosure;

FIG. 8A is a schematic diagram showing the structure of a finger according to an embodiment of this disclosure;

FIG. 8B is a schematic diagram showing an embodiment of performing the principal components analysis on the bone from the CT (computed tomography) images;

FIG. 8C is a schematic diagram showing an embodiment of performing the principal components analysis on the skin from the CT (computed tomography) images;

FIG. 8D is a schematic diagram showing an embodiment of calculating a distance between the main axis of bone and the medical instrument;

FIG. 8E is a schematic diagram showing an artificial medical image according to an embodiment of this disclosure;

FIG. 9A is a block diagram of generating an artificial medical image according to an embodiment of this disclosure;

FIG. 9B is a schematic diagram showing an artificial medical image according to an embodiment of this disclosure;

FIGS. 10A and 10B are schematic diagrams showing the calibrations of the hand phantom model and the ultrasound volume according to an embodiment of this disclosure;

FIG. 10C is a schematic diagram showing a ultrasound volume and a collision detection according to an embodiment of this disclosure;

FIG. 10D is a schematic diagram showing an artificial ultrasound image according to an embodiment of this disclosure;

FIGS. 11A and 11B are schematic diagrams showing the operation training system according to an embodiment of this disclosure; and

FIGS. 12A and 12B are schematic diagrams showing the images of the training system according to an embodiment of this disclosure.

### DETAILED DESCRIPTION OF THE DISCLOSURE

The present disclosure will be apparent from the following detailed description, which proceeds with reference to the accompanying drawings, wherein the same references relate to the same elements.

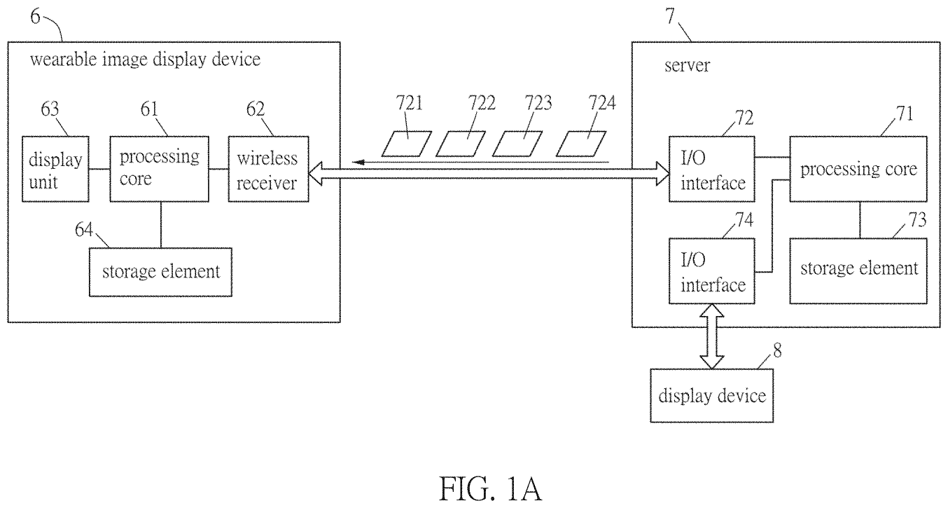

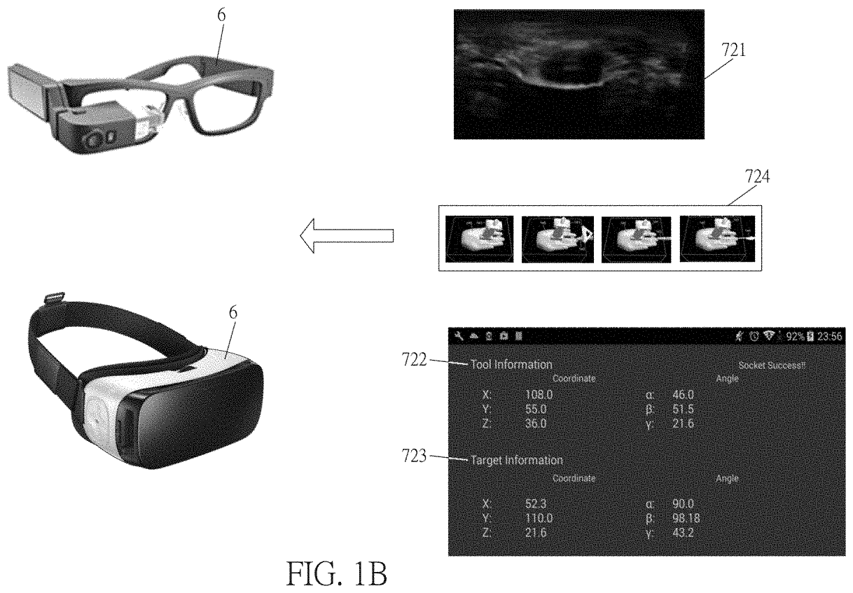

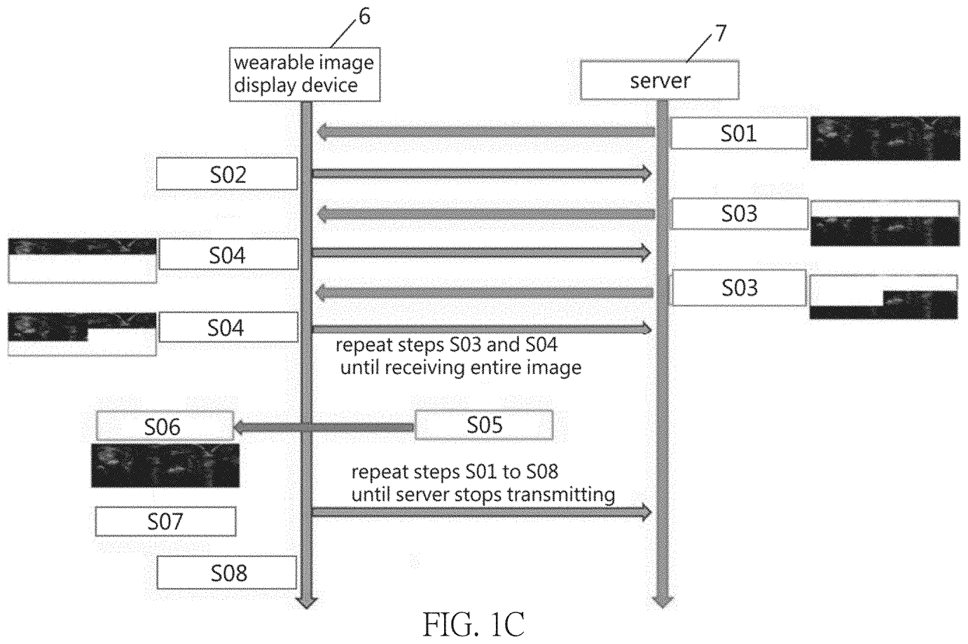

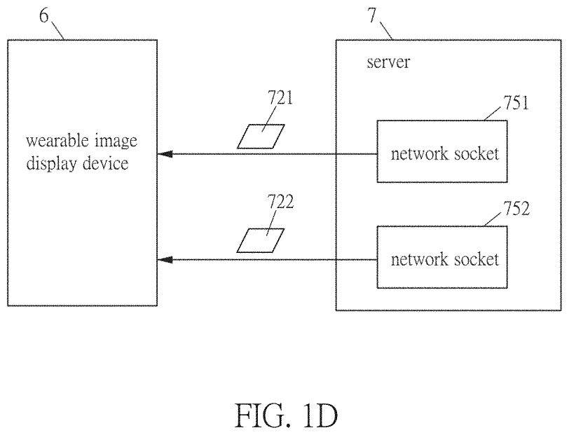

FIG. 1A is a block diagram of a surgery information real-time display system according to an embodiment of this disclosure. As shown in FIG. 1A, the surgery information real-time display system comprises a wearable image display device 6 for surgery and a server 7. The display device 6 comprises a processing core 61, a wireless receiver 62, a display unit 63, and a storage element 64. The wireless receiver 62 receives a medical image 721 or a medical instrument information 722 in real-time. The processing core 61 is coupled to the wireless receiver 62 and the display unit 63 for displaying the medical image 721 or the medical instrument information 722 on the display unit 63. The server 7 comprises a processing core 71, an I/O (input/output) interface 72, an I/O interface 74, and a storage element 73. The processing core 71 is coupled with the I/O interface 72, the I/O interface 74 and the storage element 73. The server 7 is wirelessly connected with the wireless receiver 62 for wirelessly transmitting the medical image 721 and the medical instrument information 722 in real-time. In addition, the surgery information real-time display system further comprises a display device 8, and the server 7 can output the information to the display device 8 through the I/O interface 74 for displaying the image on the display device 8.

For example, each of the processing cores 61 and 71 can be a processor, a controller, or the likes. The processor may comprise one or more cores. The processor can be a central processing unit or a graphics processing unit, and each of the processing cores 61 and 71 can also be the core of a processor or a graphics processor. On the other hand, each of the processing cores 61 and 71 can also be a processing module, and the processing module comprises a plurality of processors.

The storage elements 64 and 73 stores program codes, which can be executed by the processing cores 61 and 71, respectively. Each of the storage elements 64 and 73 comprises the non-volatile memory and volatile memory. For example, the non-volatile memory can be a hard disk, a flash memory, a solid state disk, a compact disk, and the likes, and the volatile memory can be a dynamic random access memory, a static random access memory, or the likes. For example, the program codes are stored in the non-volatile memory, and the processing cores 61 and 71 loads the program codes from the non-volatile memory into the volatile memory and then executes the program codes.

In addition, the wireless receiver 62 can wirelessly receive a surgery target information 723 in real-time, and the processing core 61 displays the medical image 721, the medical instrument information 722, or the surgery target information 723 on the display unit 63. Moreover, the wireless receiver 62 can wirelessly receive a surgery guidance video 724 in real-time, and the processing core 61 displays the medical image 721, the medical instrument information 722, or the surgery guidance video 724 on the display unit 63. The medical image, the medical instrument information, the surgery target information, or the surgery guidance video can be used to guide or prompt the user to perform the next step.

The wireless receiver 62 and the I/O interface 72 can be wireless transceivers, which complies with wireless transmission protocols such as wireless network or Bluetooth protocols. The real-time transmission method is, for example, the wireless network transmission, Bluetooth transmission, or the likes. This embodiment uses the wireless network transmission, and the wireless network is, for example, a Wi-Fi standard or a specification conforming to IEEE 802.11b, IEEE 802.11g, IEEE 802.11n, and/or the likes.

FIG. 1B is a schematic diagram showing that the wearable image display device for surgery of FIG. 1A receives the medical image or the medical instrument information. As shown in FIG. 1B, the wearable image display device is a smart glasses or a head mounted display. The smart glasses is a wearable computer glasses, which can provide additional information for the user. In addition, the smart glasses (wearable computer glasses) can change the optical property of the glasses. In general, the smart glasses can superimpose the information to the vision of the user, and the smart glasses can be used in a hands-free application. To superimpose the information to the vision can be achieved by the following technologies: the optical head-mounted display (OHMD), the embedded wireless glasses with transparent heads-up display (HUD), the augmented reality (AR), or the likes. The hands-free application can be carried out by a voice system that communicates with the smart glasses using natural language voice commands. The ultrasonic images are transmitted to the smart glasses and displayed on the smart glasses, so that the user can view the displayed images without turning head to watch another display screen.

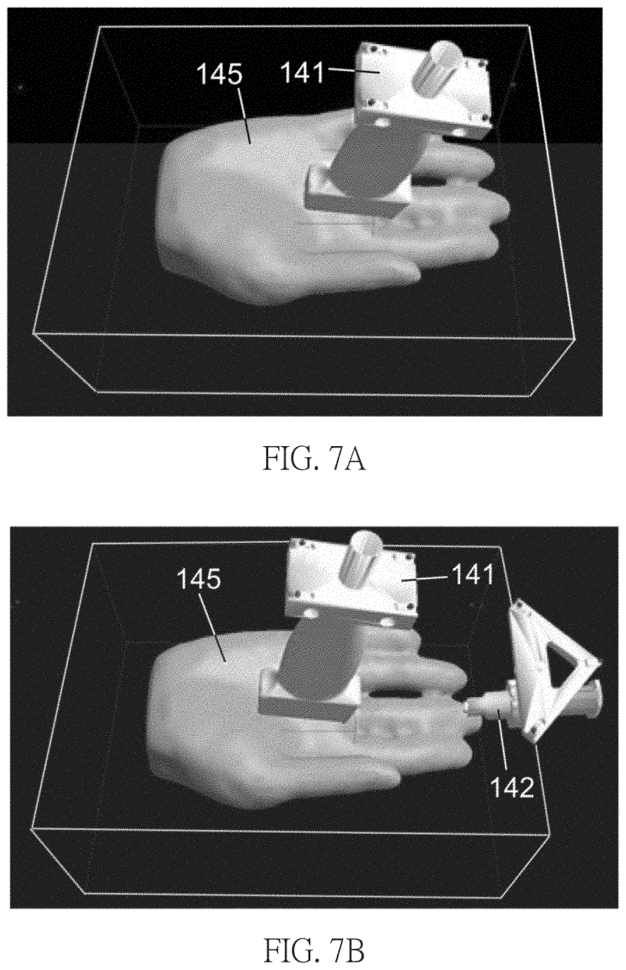

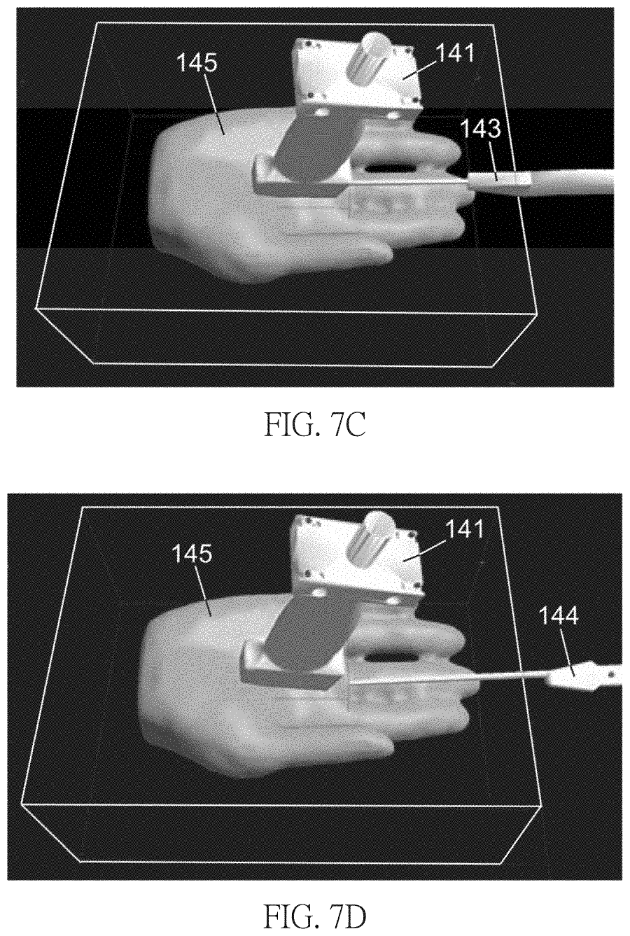

The medical image 721 is an artificial medical image of an artificial limb. For example, the artificial medical image is a medical image generated based on an artificial limb, and the medical image is an ultrasonic image. The medical instrument information 722 includes a location information and an angle information. For example, in the tool information as shown in FIG. 1B, the location information includes the X, Y, Z coordinate positions, and the angle information includes the angles a, 13, y. The surgery target information 723 includes a location information and an angle information, such as the target information as shown in FIG. 1B. The location information includes the X, Y, Z coordinate positions, and the angle information includes the angles α, β, γ. The contents of the surgery guidance video 724 can be referred to FIGS. 7A to 7D, which present the medical instruments and operations used at various stages of the surgery procedure.

In addition, the display device 6 can comprise a sound input element such as a microphone that can be used in the aforementioned hands-free application. The user can speak to issue a voice command to the display device 6 for controlling the operation of the display device 6. For example, the voice command can control to start or stop all or parts of the operation described below. This facilitates the surgery operation, and the user can control the display device 6 without putting down the instrument held in the hand. In the hands-free application, the screen of the display device 6 can display a graphic to indicate that the current voice operation mode.

FIG. 1C is a schematic diagram showing the transmission between the server and the wearable image display device for surgery of FIG. 1A. As shown in FIG. 1C, the transmission between the server 7 and the display device 6 comprises the following steps S01 to S08. In the step S01, the server 7 transmits image size information to the display device 6. In the step S02, the display device 6 replies the receiving confirmation after receiving the image size information. In the step S03, the server 7 sequentially transfers the image to the display device 6 in multiple parts. In the step S04, the display device 6 replies the receiving confirmation after receiving the image in multiple parts. The steps S03 and S04 are continuously repeated until the display device 6 has received the entire image. In the step S05, after the display device 6 receives the entire image, the display device 6 starts to process the image. Since the bmp format is a too large image format for real-time transmission, the server 7 can compress the image from the bmp format to the JPEG format to reduce the file size of the image. In the step S06, the display device 6 combines the multiple parts of the image to obtain the entire JPEG image. Then, the step S07 is to decompress and display the JPEG image, and then the transmission of the image is completed in the step S08. The steps S01 to S08 will be continuously repeated until the server 7 stops transmitting.

FIG. 1D is a schematic diagram showing that the server of FIG. lA transmits through two network sockets. As shown in FIG. 1D, in order to achieve real-time transmission of images, the server 7 transmits the medical image 721 and the medical instrument information 722 through two network sockets 751, 752, respectively. The network socket 751 is responsible for transmitting the medical image 721, and the network socket 752 is responsible for transmitting the medical instrument information 722. The display device 6 is a client, which is responsible for receiving the medical image 721 and the medical instrument information 722 from the network sockets. Compared with the general application programming interface (API) transmission, the customized socket server and client can reduce complex functions and directly transmit all data as bits arrays. In addition, the surgery target information 723 can be transmitted to the display device 6 through the network socket 751 or the additional network socket 752, and the surgery guidance video 724 can be transmitted to the display device 6 through the network socket 751 or the additional network socket 752.

Moreover, the surgery information real-time display system further comprises an optical positioning device, which detects a position of a medical instrument for generating a positioning signal. The server generates the medical instrument information according to the positioning signal. The optical positioning device comprises, for example, optical markers and optical sensor of the subsequent embodiment. The surgery information real-time display system can be used in the optical tracking system and the training system of the following embodiments. The display device 8 can be the output device 5 of the following embodiments. The server can be the computing device 13 of the following embodiments. The I/O interface 74 can be the I/O interface 134 of the following embodiments, and the I/O interface 72 can be the I/O interface 137 of the following embodiments. The content output by the I/O interface 134 of the following embodiments may also be converted to the relevant format and then transmitted to the display device 6 through the I/O interface 137, thereby the display device 6 can display the received content.

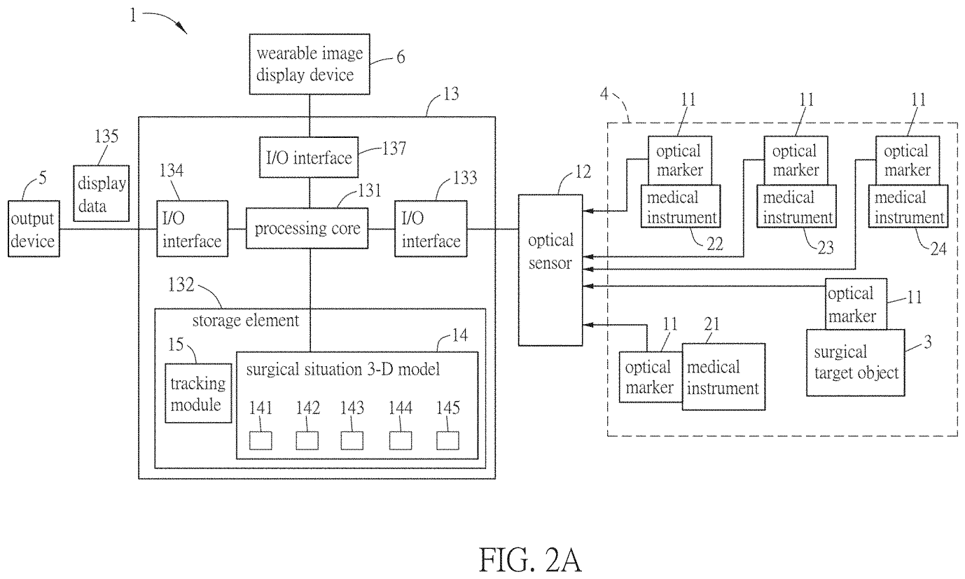

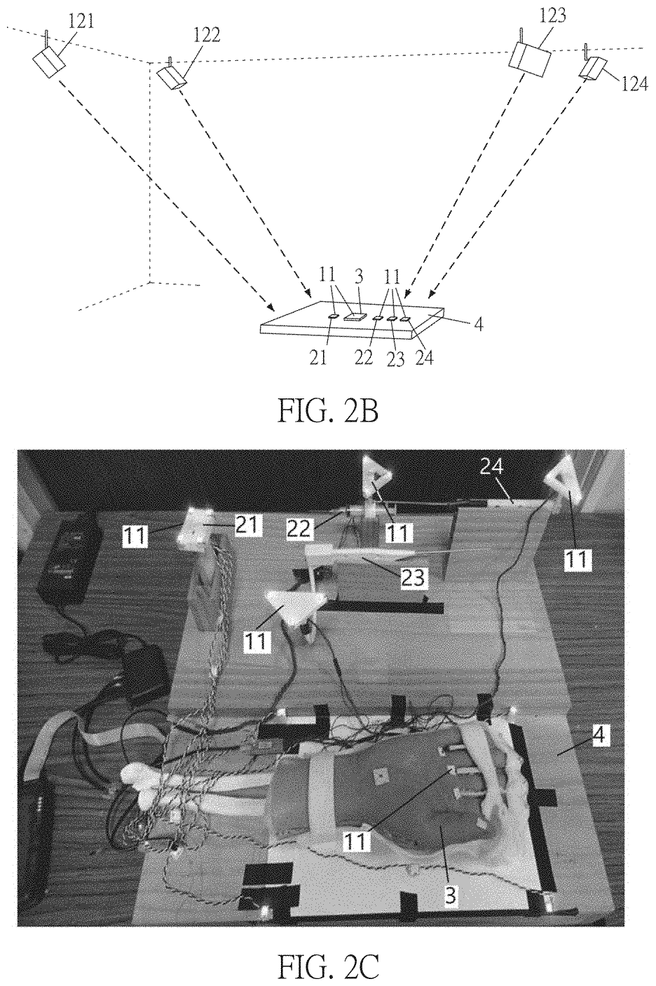

FIG. 2A is a block diagram showing an optical tracking system according to an embodiment of this disclosure. As shown in FIG. 2A, an optical tracking system 1 for a medical instrument comprises a plurality of optical markers 11, a plurality of optical sensors 12, and a computing device 13. The optical markers 11 are disposed on one or more medical instruments. In this embodiment, for example, the optical markers 11 are disposed on multiple medical instruments 21˜24. In addition, the optical markers 11 can also be disposed on a surgical target object 3, and the medical instruments 21˜24 and the surgical target object 3 are placed on a platform 4. The optical sensors 12 optically sense the optical markers 11 to respectively generate a plurality of sensing signals. The computing device 13 is coupled to the optical sensors 12 for receiving the sensing signals. The computing device 13 comprises a surgical situation 3-D model 14, and is configured to adjust a relative position between virtual medical instrument objects 141˜144 and a virtual surgical target object 145 in the surgical situation 3-D model 14 according to the sensing signals. Referring to FIG. 2D, the virtual medical instrument objects 141˜144 and the virtual surgical target object 145 represent the medical instruments 21˜24 and the surgical target object 3 in the surgical situation 3-D model 14. In the optical tracking system 1, the surgical situation 3-D model 14 can obtain the current positions of the medical instrument 21˜24 and the surgical target object 3, which can reflect to the virtual medical instrument objects and the virtual surgical target object.

The optical tracking system 1 comprises at least two optical sensors 12, which are disposed above the medical instruments 21˜24 and toward the optical markers 11 for real-time tracking the medical instruments 21˜24 so as to obtain the positions thereof. The optical sensors 12 can be camera-based linear detectors. FIG. 2B is a schematic diagram showing the optical tracking system according to an embodiment of this disclosure. For example, as shown in FIG. 2B, four optical sensors 121˜124 are installed on the ceiling and disposed toward the optical markers 11, the medical instruments 21˜24 and the surgical target object 3 on the platform 4.

For example, the medical instrument 21 is a medical detection tool such as a probe for ultrasonic image detection or any device that can detect the internal structure of the surgical target object 3. These devices are used clinically, and the probe for ultrasonic image detection is, for example, an ultrasonic transducer. The medical instruments 22˜24 are surgical instruments such as needles, scalpels, hook blades, and the likes, which are clinically used. If used for surgical training, the medical detection tool can be a clinically used device or a simulated virtual clinical device, and the surgical tool can also be a clinically used device or a simulated virtual clinical device. For example, FIG. 2C is a schematic diagram of an optical tracking system of an embodiment. As shown in FIG. 2C, the medical instruments 21˜24 and the surgical target object 3 on the platform 4 are used for surgical training, such as finger minimally invasive surgery, which can be used for the trigger finger surgery. The clippers of the platform 4 and the medical instruments 21˜24 may be made of woody material. The medical instrument 21 is an immersive ultrasonic transducer (or probe), and the medical instruments 22˜24 include a plurality of surgical instruments, such as dilators, needles, and hook blades. The surgical target object 3 is a hand phantom. Each of the medical instruments 21˜24 is configured with three or four optical markers 11, and the surgical target object 3 is also configured with three or four optical markers 11. For example, the computing device 13 is connected to the optical sensors 12 for tracking the positions of the optical markers 11 in real time. In this embodiment, there are 17 optical markers 11, including 4 optical markers 11 located on or around the surgical target object 3 and moved relative to the surgical target object 3, and 13 optical markers 11 on the medical instruments 21˜24. The optical sensors 12 continuously transmits the real-time information to the computing device 13. In addition, the computing device 13 also uses the motion judging function to reduce the calculation loading. If the moving distance of the optical marker 11 is less than a threshold value, the position of the optical marker 11 is not updated. The threshold value is, for example, 0.7 mm.

Referring to FIG. 2A, the computing device 13 includes a processing core 131, a storage element 132, and a plurality of I/O interfaces 133 and 134. The processing core 131 is coupled to the storage element 132 and the I/O interfaces 133 and 134. The I/O interface 133 can receive the sensing signals generated by the optical sensors 12, and the I/O interface 134 communicates with the output device 5. The computing device 13 can output the processing result to the output device 5 through the I/O interface 134. The I/O interfaces 133 and 134 are, for example, peripheral transmission ports or communication ports. The output device 5 is a device capable of outputting images, such as a display, a projector, a printer, and the likes.

The storage element 132 stores program codes, which can be executed by the processing core 131. The storage element 132 comprises the non-volatile memory and volatile memory. For example, the non-volatile memory can be a hard disk, a flash memory, a solid state disk, a compact disk, and the likes, and the volatile memory can be a dynamic random access memory, a static random access memory, or the likes. For example, the program codes are stored in the non-volatile memory, and the processing core 131 loads the program code from the non-volatile memory into the volatile memory and then executes the program code. The storage element 132 stores the program codes and data of the surgical situation 3-D model 14 and the tracking module 15. The processing core 131 can access the storage element 132 to execute and process the program codes and data of the surgical situation 3-D model 14 and the tracking module 15.

The processing core 131 can be, for example, a processor, a controller, or the likes. The processor may comprise one or more cores. The processor can be a central processing unit or a graphics processing unit, and the processing core 131 can also be the core of a processor or a graphics processor. On the other hand, the processing core 131 can also be a processing module, and the processing module comprises a plurality of processors.

The operation of the optical tracking system includes a connection between the computing device 13 and the optical sensors 12, a pre-operation process, a coordinate calibration process of the optical tracking system, a rendering process, and the likes. The tracking module 15 represents the relevant program codes and data of these operations. The storage element 132 of the computing device 13 stores the tracking module 15, and the processing core 131 executes the tracking module 15 to perform these operations.

The computing device 13 can perform the pre-operation and the coordinate calibration of the optical tracking system to find the optimum transform parameter, and then the computing device 13 can set positions of the virtual medical instrument objects 141˜144 and the virtual surgical target object 145 in the surgical situation 3-D model 14 according to the optimum transform parameter and the sensing signals. The computing device 13 can derive the positions of the medical instrument 21 inside and outside a surgical target object 3, and adjusts the relative position between the virtual medical instrument objects 141˜144 and the virtual surgical target object 145 in the surgical situation 3-D model 14. Accordingly, the medical instruments 21˜24 can be real-time tracked from the detection result of the optical sensors 12 and correspondingly presented in the surgical situation 3-D model 14. The virtual objects (representations) in the surgical situation 3-D model 14 are as shown in FIG. 2D.

The surgical situation 3-D model 14 is a native model, which comprises the model established for the surgical target object 3 as well as the model established for the medical instruments 21˜24. For example, the developer can establish the model on a computer by computer graphic technology. In practice, the user may operate a graphic software or a specific software to establish the models.

The computing device 13 can output the display data 135 to the output device 5 for displaying 3-D images of the virtual medical instrument objects 141˜144 and the virtual surgical target object 145. The output device 5 can output the display data 135 by displaying, printing, or the likes. FIG. 2D shows that the display data 135 is outputted by displaying.

The coordinate position of the surgical situation 3-D model 14 can be accurately transformed to the corresponding optical marker 11 in the tracking coordinate system, and vice versa. Thereby, the medical instruments 21˜24 and the surgical target object 3 can be tracked in real-time based on the detection result of the optical sensors 12, and the positions of the medical instruments 21˜24 and the surgical target object 3 in the tracking coordinate system are processed through the aforementioned processing, thereby correspondingly showing the virtual medical instrument objects 141˜144 and the virtual surgical target object 145 in the surgical situation 3-D model 14. When the medical instruments 21˜24 and the surgical target object 3 physically move, the virtual medical instrument objects 141˜144 and the virtual surgical target object 145 will correspondingly move in the surgical situation 3-D model 14 in real-time.

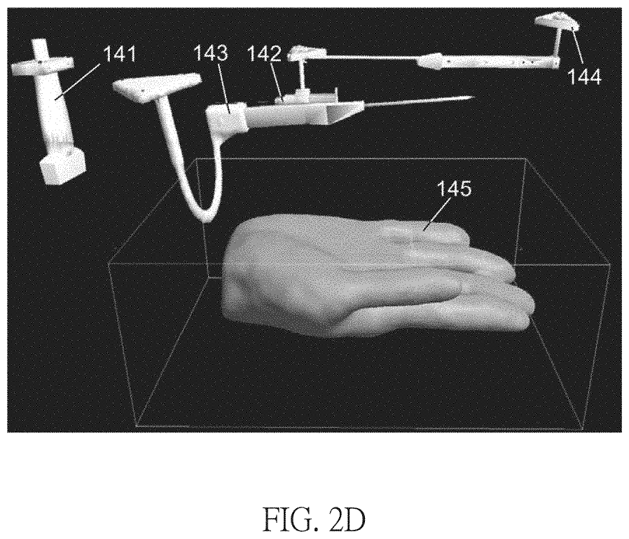

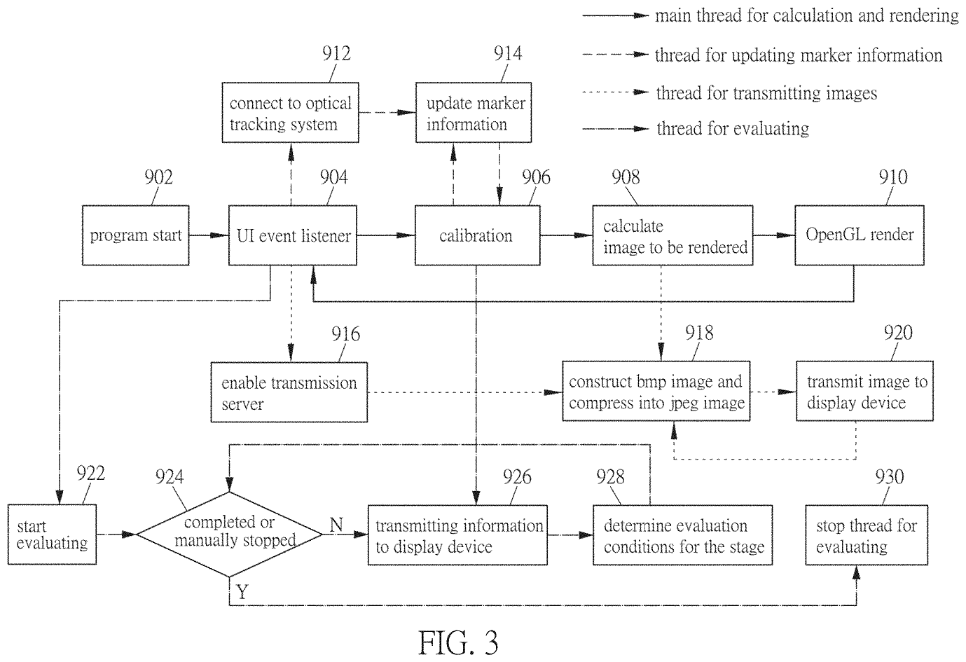

FIG. 3 is a block diagram of a surgery training system according to an embodiment of this disclosure. As shown in FIG. 3, the surgery information real-time display system can be used in the surgery training system, and the server 7 can perform the blocks shown in FIG. 3. In order to achieve real-time processing, multiple functions can be programmed to execute in multiple threads. For example, FIG. 3 shows four threads, which are the main thread for calculation and rendering, the thread for updating marker information, the thread for transmitting images, and the thread for evaluating.

The main thread for calculation and rendering comprises blocks 902 to 910. At the block 902, the program of the main thread starts executing. At the block 904, the UI event listener opens other threads for the event or further executes other blocks of the main thread. At block 906, the optical tracking system is calibrated, and then the image to be rendered is calculated at block 908. Afterwards, at the block 910, the image is rendered by OpenGL.

The thread for updating marker information comprises blocks 912 to 914. The thread for updating marker information is opened by block 904. At the block 912, the server 7 is connected to the component of the optical tracking system, such as an optical sensor. Afterwards, the marker information is updated at the block 914. Between the block 914 and the block 906, the two threads share the memory to update the marker information.

The thread for transmitting images comprises the blocks 916 to 920. The thread for transmitting images is opened from the block 904. At the block 916, the transmission server is enabled. Then, at the block 918, the image rendered from the block 908 is constructed to a bmp image and then compressed into a JPEG image. Afterwards, at the block 920, the image is transmitted to the display device.

The thread for evaluating comprises the blocks 922 to 930. The thread for evaluating is opened by block 904 and starts from the block 922. The block 924 determines that the training stage is completed or manually stopped. If the training stage is completed, the block 930 is performed to stop the thread for evaluating, and if the training stage is manually stopped by the trainee, the block 926 is performed. At the block 926, the marker information is obtained from the block 906 and the current training stage information is transmitted to the display device. The block 928 determines the evaluation conditions for the stage, and then the procedure returns to the block 924.

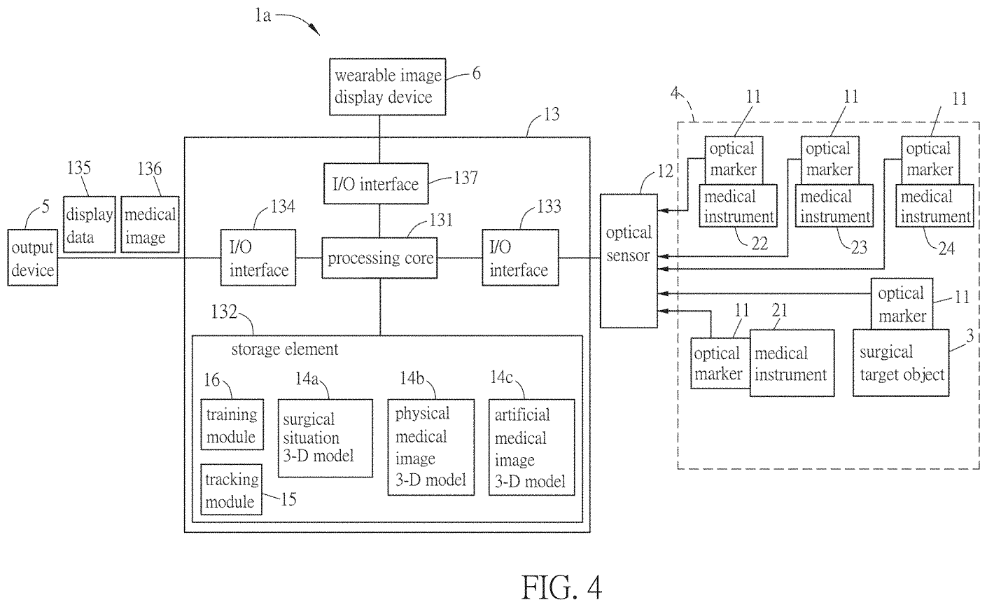

FIG. 4 is a block diagram showing a training system for operating a medical instrument according to an embodiment of this disclosure. A training system for operating a medical instrument can realistically simulate a surgical training situation. The training system comprises an optical tracking system la, one or more medical instruments 21˜24, and a surgical target object 3. The optical tracking system la includes a plurality of optical markers 11, a plurality of optical sensors 12, and a computing device 13. The optical markers 11 are disposed on the medical instruments 21˜24 and the surgical target object 3, and the medical instruments 21˜24 and the surgical target object 3 are placed on the platform 4. Corresponding to the medical instruments 21˜24 and the surgical target object 3, the virtual medical instrument objects 141˜144 and the virtual surgical target object 145 are correspondingly presented in the surgical situation 3-D model 14a. The medical instruments 21˜24 include medical detection tools and surgical tools. For example, the medical instrument 21 is a medical detection tool (probe), and the medical instruments 22˜24 are surgical tools. The virtual medical instrument objects 141˜144 include medical detection virtual tools and surgical virtual tools. For example, the virtual medical instrument object 141 is a medical detection virtual tool, and the virtual medical instrument objects 142˜144 are surgical virtual tools. The storage element 132 stores the program codes and data of the surgical situation 3-D model 14a and the tracking module 15. The processing core 131 can access the storage element 132 to execute and process the program codes and data of the surgical situation 3-D model 14a and the tracking module 15. The implementations and variations of the corresponding elements having the same reference numbers in the above description and related drawings may be referred to the description of the above embodiment, and thus will not be described again.

The surgical target object 3 can be an artificial limb, such as upper limb phantom, hand phantom, palm phantom, finger phantom, arm phantom, upper arm phantom, forearm phantom, elbow phantom, upper limb phantom, feet phantom, toes phantom, ankles phantom, calves phantom, thighs phantom, knees phantom, torso phantom, neck phantom, head phantom, shoulder phantom, chest phantom, abdomen phantom, waist phantom, hip phantom or other phantom parts, etc.

In this embodiment, the training system is applied for training, for example, the minimally invasive surgery of finger. In this case, the surgical target object 3 is a hand phantom, and the surgery is, for example, a trigger finger surgery. The medical instrument 21 is an immersive ultrasonic transducer (or probe), and the medical instruments 22˜24 are a needle, a dilator, and a hook blade. In other embodiments, the surgical target object 3 can be different parts for performing other surgery trainings.

The storage element 132 further stores the program codes and data of a physical medical image 3-D module 14b, an artificial medical image 3-D module 14c, and a training module 16. The processing core 131 can access the storage element 132 to execute and process the program codes and data of the physical medical image 3-D module 14b, the artificial medical image 3-D module 14c, and the training module 16. The training module 16 responses for performing the following surgery training procedures and the processing, integrating and calculating of the related data.

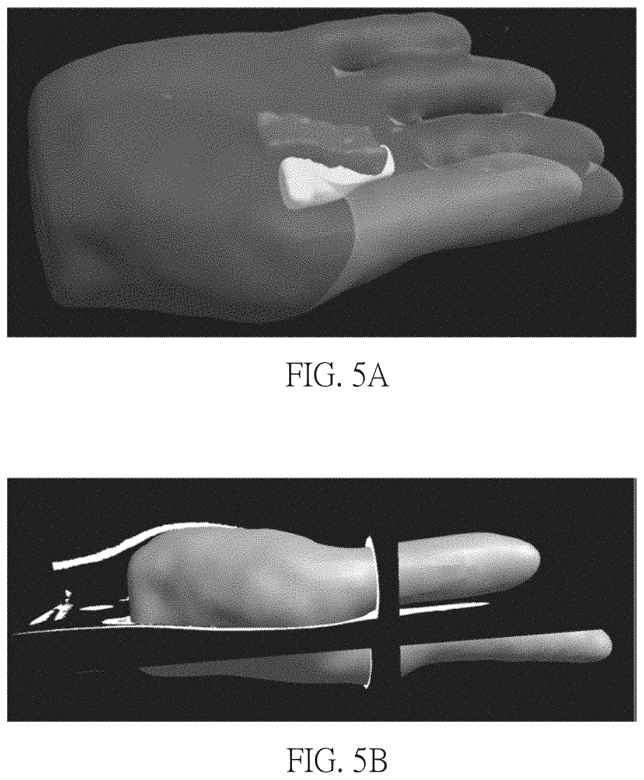

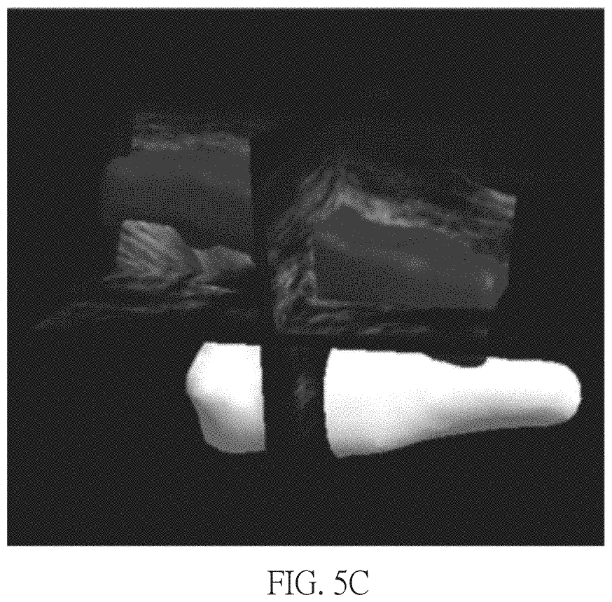

The image model for surgery training is pre-established and imported into the system prior to the surgery training process. Taking the finger minimally invasive surgery as an example, the image model includes finger bones (palm and proximal phalanx) and flexor tendon. These image models can refer to FIGS. 5A to 5C. FIG. 5A a schematic diagram showing a surgical situation 3-D model according to an embodiment of this disclosure, FIG. 5B is a schematic diagram showing a physical medical image 3-D model according to an embodiment of this disclosure, and FIG. 5C is a schematic diagram showing an artificial medical image 3-D model according to an embodiment of this disclosure. The contents of these 3-D models can be outputted or printed by the output device 5.

The physical medical image 3-D model 14b is a 3-D model established from the medical image, and it is established for the surgical target object 3 (e.g. the 3-D model of FIG. 5B). The medical images can be, for example, the CT (computed tomography) images, which is obtained by subjecting the surgical target object 3 to the computed tomography. The obtained CT images can be used to establish the physical medical image 3-D model 14b.

The artificial medical image 3-D model 14c contains an artificial medical image model, which is established for the surgical target object 3, such as the 3-D model as shown in FIG. 5C. For example, the artificial medical image model is a 3-D model of an artificial ultrasound image. Since the surgical target object 3 is not a real life body, the computed tomography can obtain a physical structural images, but other medical image equipment such as ultrasonic image equipment cannot obtain the effective or meaningful images directly from the surgical target object 3. Therefore, the ultrasonic image model of the surgical target object 3 must be produced in an artificial manner. In practice, an appropriate position or plane is selected from the 3-D model of the artificial ultrasound image so as to generate a 2-D artificial ultrasound image.

The computing device 13 generates a medical image 136 according to the surgical situation 3-D model 14a and the medical image model. The medical image model is, for example, the physical medical image 3-D model 14b or the artificial medical image 3-D model 14c. For example, the computing device 13 generates a medical image 136 according to the surgical situation 3-D model 14a and the artificial medical image 3-D model 14c. Herein, the medical image 136 is a 2-D artificial ultrasound image. The computing device 13 evaluates a score according to a process of utilizing the medical detection virtual tool 141 to find a detected object and an operation of the surgical virtual tool 145. Herein, the detected object is, for example, a specific surgical site.

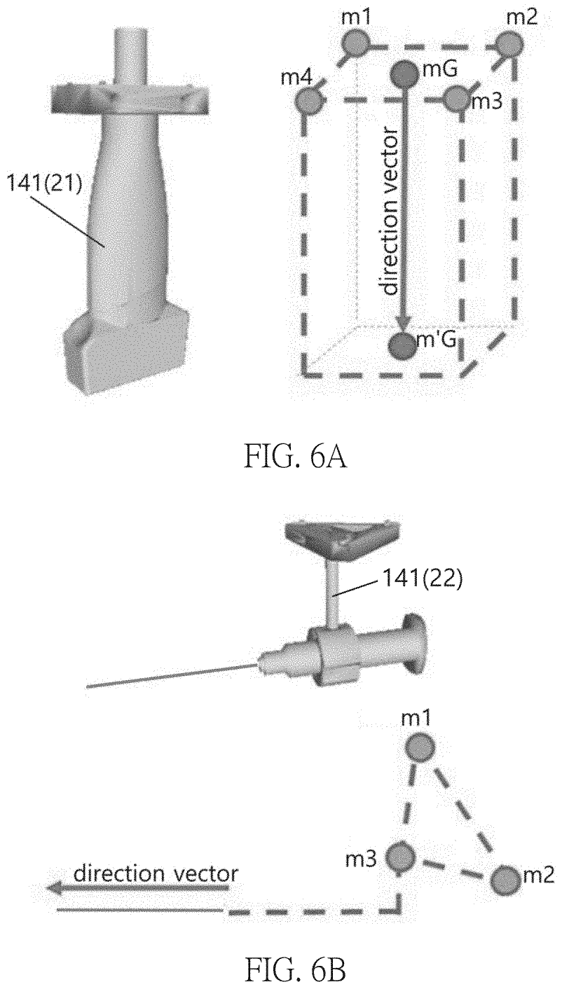

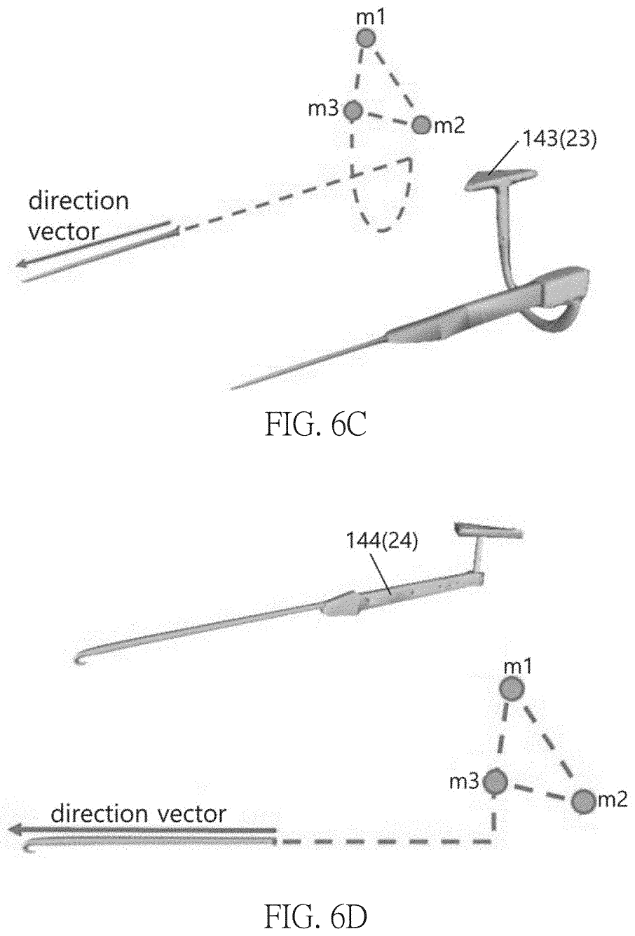

FIGS. 6A to 6D are schematic diagrams showing direction vectors of the medical instruments according to an embodiment of this disclosure. The direction vectors of the virtual medical instrument objects 141˜144 corresponding to the medical instrument 21˜24 can be rendered in real-time. Regarding the virtual medical instrument object 141, the direction vector of the medical detection tool can be obtained by calculating the center of weight of the optical marker, and another point is projected to the x-z plane so as to calculate the vector from the center of weight to the projection point. Regarding the other virtual medical instrument objects 142˜144, the direction vectors thereof can be easily calculated based on the sharp points in the model.

In order to reduce the system loading and avoid delays, the amount of image depiction can be reduced. For example, the training system can only draw the model in the area where the virtual surgical target object 145 is located rather than all of the virtual medical instrument objects 141˜144.

In the training system, the transparency of the skin model can be adjusted to observe the anatomy inside the virtual surgical target object 145, and to view an ultrasound image slice or a CT image slice of a different cross section, such as a horizontal plane (axial plane), a sagittal plane, or coronal plane. This configuration can help the surgeon during the operation. The bounding boxes of each model are constructed for collision detection. The surgery training system can determine which medical instrument has contacted the tendons, bones and/or skin, and can determine when to start evaluation.

Before the calibration process, the optical markers 11 attached to the surgical target object 3 must be clearly visible or detected by the optical sensor 12. The accuracy of detecting the positions of the optical markers 11 will decrease if the optical markers 11 are shielded. The optical sensor 12 needs to sense at least two whole optical markers 11. The calibration process is as described above, such as a three-stage calibration, which is used to accurately calibrate two coordinate systems. The calibration error, the iteration count, and the final positions of the optical markers can be displayed in a window of the training system, such as the monitor of the output device 5. Accuracy and reliability information can be used to alert the user that the system needs to be recalibrated when the error is too large. After the coordinate system calibration is completed, the 3-D model is drawn at a frequency of 0.1 times per second, and the rendered result can be output to the output device 5 for displaying or printing.

After preparing the training system, the user can start the surgery training procedure. In the training procedure, the first step is to operate the medical detection tool to find the surgery site, and then the site will be anesthetized. Afterward, the path from the outside to the surgery site is expanded, and then the scalpel can reach the surgery site through the expanded path.

FIGS. 7A to 7D are schematic diagrams showing the training procedure of the training system according to an embodiment of this disclosure.

As shown in FIG. 7A, in the first stage, the medical detection tool 21 is used to find the surgery site to confirm that the site is within the training system. The surgery site is, for example, a pulley, which can be judged by finding the positions of the metacarpal joints (MCP joints), the bones of the fingers, and the anatomy of the tendon. The point of this stage is whether the first pulley (A1 pulley) is found or not. In addition, if the trainee does not move the medical detection tool for more than three seconds to determine the position, then the training system will automatically proceed to the evaluation of next stage. During the surgical training, the medical detection tool 21 is placed on the skin and remained in contact with the skin at metacarpal joints (MCP joints) on the midline of the flexor tendon.

As shown in FIG. 7B, in the second stage, the surgical instrument 22 is used to open the path of the surgical field, and the surgical instrument 22 is, for example, a needle. The needle is inserted to inject a local anesthetic and expand the space, and the insertion of the needle can be performed under the guidance of a continuous ultrasound image. This continuous ultrasound image is an artificial ultrasound image, such as the aforementioned medical image 136. Because it is difficult to simulate local anesthesia of a hand phantom, no special simulation of anesthesia is conducted.

As shown in FIG. 7C, in the third stage, the surgical instrument 23 is pushed along the same path as the surgical instrument 22 in the second stage to create the trace required for the hook blade in the next stage. The surgical instrument 23 is, for example, a dilator. In addition, if the trainee does not move the surgical instrument 23 for more than three seconds to determine the position, then the training system will automatically proceed to the evaluation of the next stage.

As shown in FIG. 7D, in the fourth stage, the surgical instrument 24 is inserted along the trace created in the third stage, and the pulley is divided by the surgical instrument 24, such as a hook blade. The point of the fourth stage is similar to that of the third stage. During the surgery training, the vessels and nerves along the two sides of the flexor tendon may be easily cut unintentionally, so the key points of the third and fourth stages are to not contact the tendons, nerves and vessels, and to open a trace that is at least 2 mm over the first pulley, thereby leaving the space for the hook blade to cut the pulley.

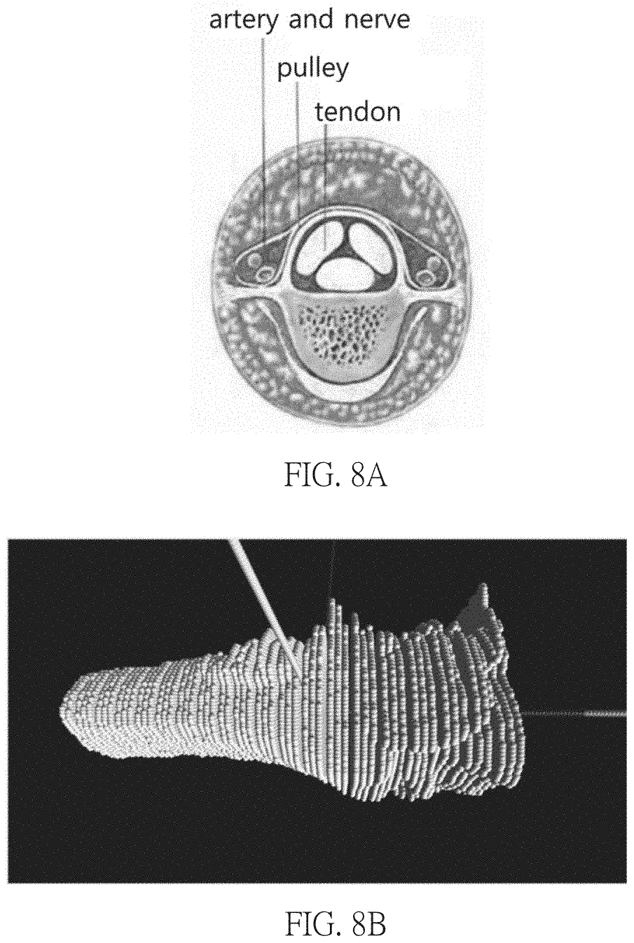

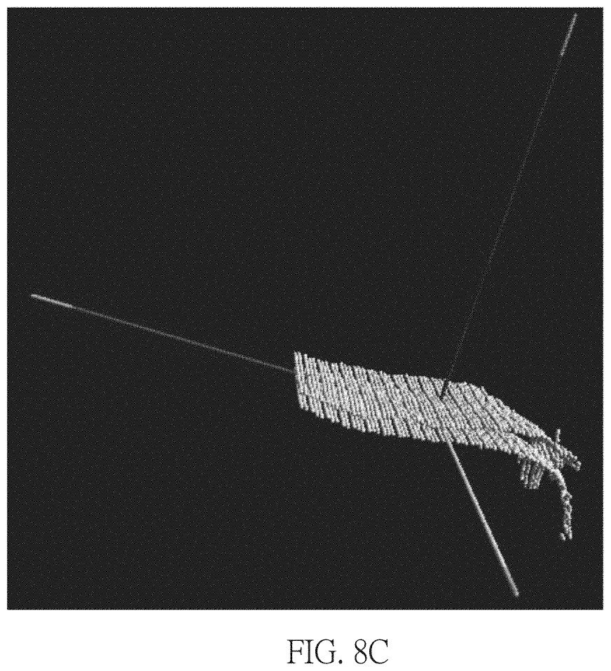

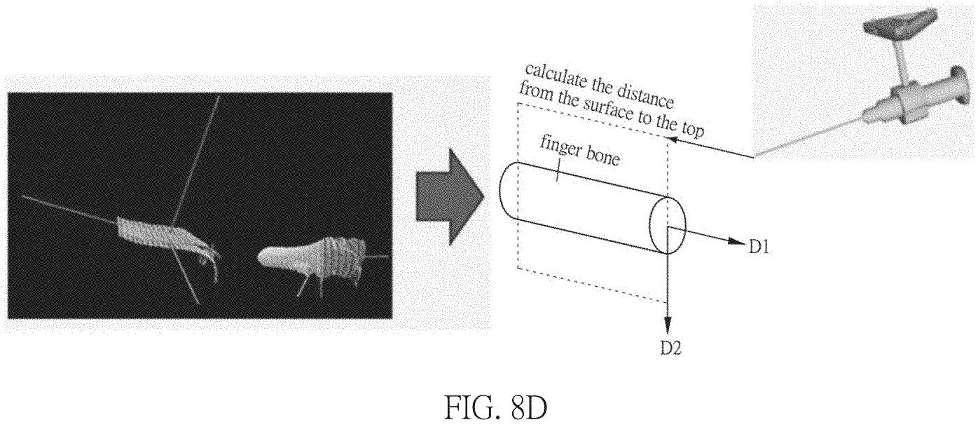

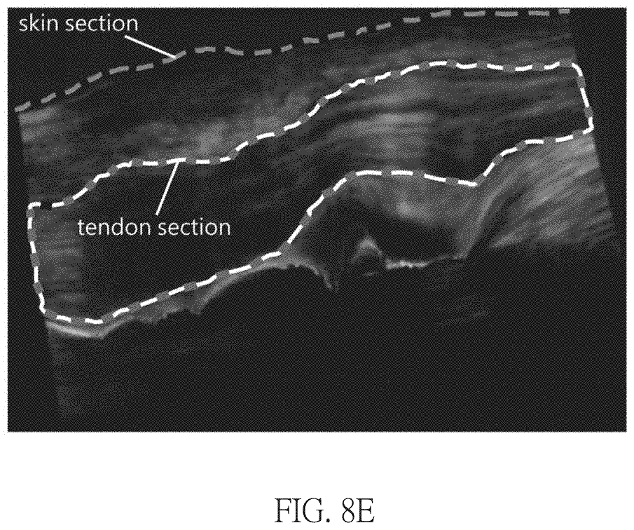

In order to evaluate the operation of the user, the operation of each training stage must be quantified. First, the surgical field in operation is defined by the finger anatomy of FIG. 8A, which can be divided into an upper boundary and a lower boundary. Since most of the tissues around the tendon are fat, it does not cause pain. Thus, the upper boundary of the surgical field can be defined by the skin of the palm, and the lower boundary can be defined by the tendon. The proximal depth boundary is 10 mm (average length of the first pulley) from the metacarpal head-neck joint. The distal depth boundary is not important because it is not associated with damages of tendon, vessels and nerves. The left and right boundaries are defined by the width of the tendon, and the nerves and vessels are located at two sides of the tendon.

After defining the surgical field, the evaluating method for each training stage is as follows. In the first stage of FIG. 7A, the point of the training is to find the target, for example, the object to be cut. Taking the finger as an example, the A1 pulley is the object to be cut. In the actual surgery procedure, in order to obtain the good ultrasound image quality, the angle between the medical detection tool and the main axis of bone should be close to vertical, and the allowable angular deviation is ±30°. Therefore, the equation of evaluating the first stage is as follow:

score of first stage=(score for finding the object)×(weight)+(score of the angle of medical detection tool)×(weight)

In the second stage of FIG. 7B, the point of the training is to use a needle to open the path of the surgical field. Since the pulley surrounds the tendon, the distance between the main axis of bone and the needle should be as small as better. Therefore, the equation of evaluating the second stage is as follow:

score of second stage=(score for opening the path)×(weight)+(score of the angle of needle)×(weight)+(score of the distance from main axis of bone)×(weight)

In the third stage, the point of the training is to insert the dilator into the finger for enlarging the surgical field. During the surgery, the trace of the dilator must be close to the main axis of bone. In order to not damage the tendon, vessels and nerves, the dilator does not exceed the boundaries of the previously defined surgical field. In order to properly expand the trace for the surgical field, the angle between the dilator and the main axis of bone is preferably approximately in parallel with an allowable angular deviation of ±30°. The dilator must be at least 2 mm over the first pulley for leaving the space for the hook blade to cut the first pulley. The equation of evaluating the third stage is as follow:

score of third stage=(score of over the pulley)×(weight)+(score of the angle of dilator)×(weight)+(score of the distance from main axis of bone)×(weight)+(score of not leaving the surgical field)×(weight)

In the fourth stage, the evaluation conditions of the fourth stage is similar to that of the third stage. Different from the third stage, the evaluation of rotating the hook blade for 90° must be added to the evaluation of the fourth stage. The equation of evaluating the fourth stage is as follow:

score of third stage=(score of over the pulley)×(weight)+(score of the angle of hook blade)×(weight)+(score of the distance from main axis of bone)×(weight)+(score of not leaving the surgical field)×(weight)+(score of rotating the hook blade)×(weight)

In order to establish the evaluating standards to evaluate the surgery operation of a user, it is necessary to define how to calculate the angle between the main axis of bone and the medical instrument. For example, this calculation is the same as calculating the angle between the palm normal and the direction vector of the medical instrument. First, the main axis of bone must be found. As shown in FIG. 8B, the three axes of the bone can be found by using Principal Components Analysis (PCA) on the bone from the computed tomography images. Among the three axes, the longest axis is taken as the main axis of bone. However, in computed tomography images, the shape of the bone is uneven, which causes that the palm normal and the axis found by PCA are not perpendicular to each other. Thus, as shown in FIG. 8C, instead of using PCA on the bone, the skin on the bone can be used to find the palm normal by using PCA. The angle between the main axis of bone and the medical instrument can then be calculated.

After calculating the angle between the main axis of bone and the medical instrument, it is also needed to calculate the distance between the main axis of bone and the medical instrument. This distance calculation is similar to calculating the distance between the top of the medical instrument and the plane. The plane refers to the plane containing the main axis of bone and the palm normal. The distance calculation is shown in FIG. 8D. This plane can be obtained by the cross product of the vector D2 of the palm normal and the vector D1 of the main axis of bone. Since these two vectors can be calculated in the previous calculation, the distance between the main axis of bone and the medical instrument can be easily calculated.

FIG. 8E is a schematic diagram showing an artificial medical image according to an embodiment of this disclosure, wherein the tendon section and the skin section in the artificial medical image are indicated by dotted lines. As shown in FIG. 8E, the tendon section and the skin section can be used to construct the model and the bounding box. The bounding box is used for collision detection, and the pulley can be defined in the static model. By using the collision detection, it is possible to determine the surgical field and judge whether the medical instrument crosses the pulley or not. The average length of the first pulley is approximately 1 mm. The first pulley is located at the proximal end of the MCP head-neck joint. The average thickness of the pulley surrounding the tendon is approximately 0.3 mm.

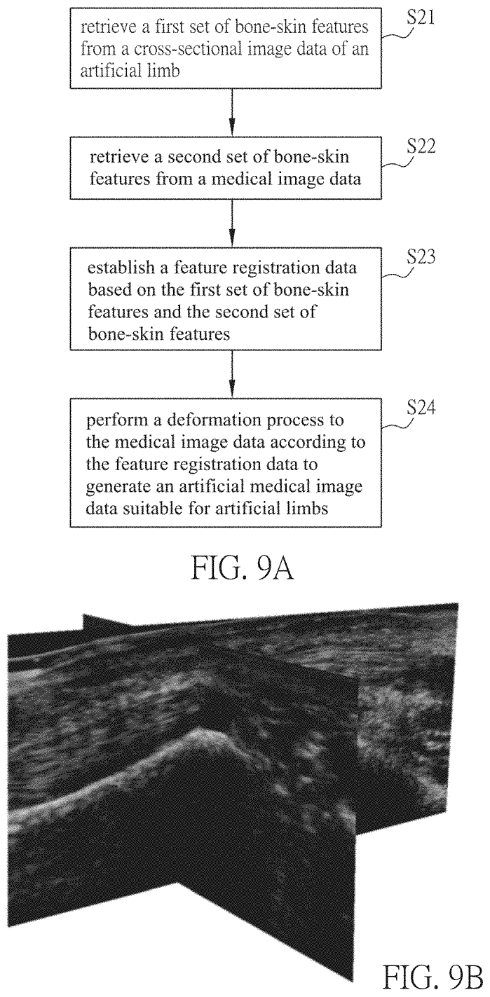

FIG. 9A is a block diagram of generating an artificial medical image according to an embodiment of this disclosure. As shown in FIG. 9A, the generating procedure comprises the steps S21 to S24.

The step S21 is to retrieve a first set of bone-skin features from a cross-sectional image data of an artificial limb. The artificial limb is the aforementioned surgical target object 3, which can be used as a limb for minimally invasive surgery training, such as a hand phantom. The cross-sectional image data contain multiple cross-sectional images, and the cross-sectional reference images are computed tomography images or physical cross-sectional images.

The step S22 is to retrieve a second set of bone-skin features from a medical image data. The medical image data is a stereoscopic ultrasound image, such as the stereoscopic ultrasound image of FIG. 9B, which is established by a plurality of planar ultrasound images. The medical image data is a medical image taken of a real creature instead of an artificial limb. The first set of bone-skin features and the second set of bone-skin features comprise a plurality of bone feature points and a plurality of skin feature points.

The step S23 is to establish a feature registration data based on the first set of bone-skin features and the second set of bone-skin features. The step S23 comprises: taking the first set of bone-skin features as the reference target; and finding a correlation function as the spatial correlation data, wherein the correlation function satisfies that when the second set of bone-skin features aligns to the reference target, there is no interference caused by the first set of bone-skin features and the second set of bone-skin features. The correlation function is found through the algorithm of the maximum likelihood estimation problem and the EM algorithm.

The step S24 is to perform a deformation process to the medical image data according to the feature registration data to generate an artificial medical image data suitable for artificial limbs. The artificial medical image data is, for example, a stereoscopic ultrasound image that maintains the features of the organism within the original ultrasound image. The step S24 comprises: generating a deformation function according to the medical image data and the feature registration data; applying a grid to the medical image data to obtain a plurality of mesh dot positions; deforming the mesh dot positions according to the deformation function; and generating a deformed image by adding corresponding pixels from the medical image data based on the deformed mesh dot positions, wherein the deformed image is used as the artificial medical image data. The deformation function is generated by moving least square (MLS). The deformed image is generated by using the affine transform.

In the steps S21 to S24, the image features are retrieved from the real ultrasound image and the computed tomography image of hand phantom, and the corresponding point relationship of the deformation is obtained by the image registration. Afterward, an artificial ultrasound image which is like an ultrasound image of human is generated by the deformation based on the hand phantom, and the generated ultrasound image can maintain the features in the original real ultrasound image. In the case that the artificial medical image data is a stereoscopic ultrasonic image, a plane ultrasonic image of a specific position or a specific slice surface can be generated according to a position or a slice surface corresponding to the stereoscopic ultrasonic image.

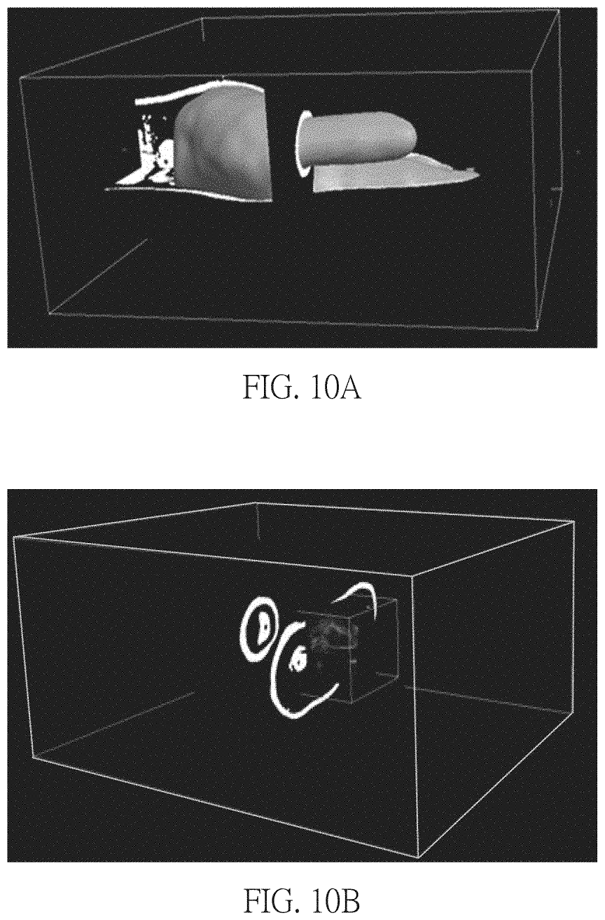

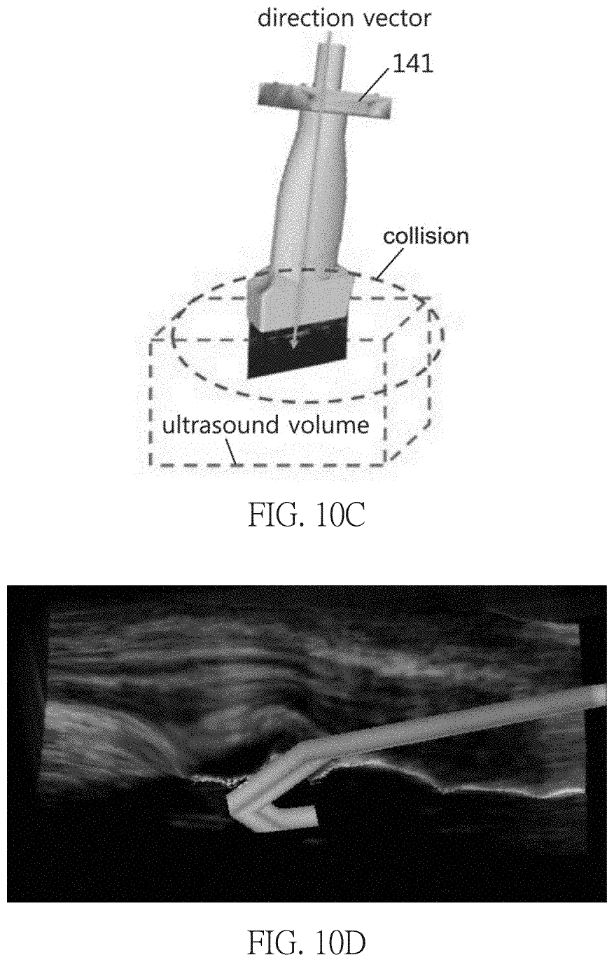

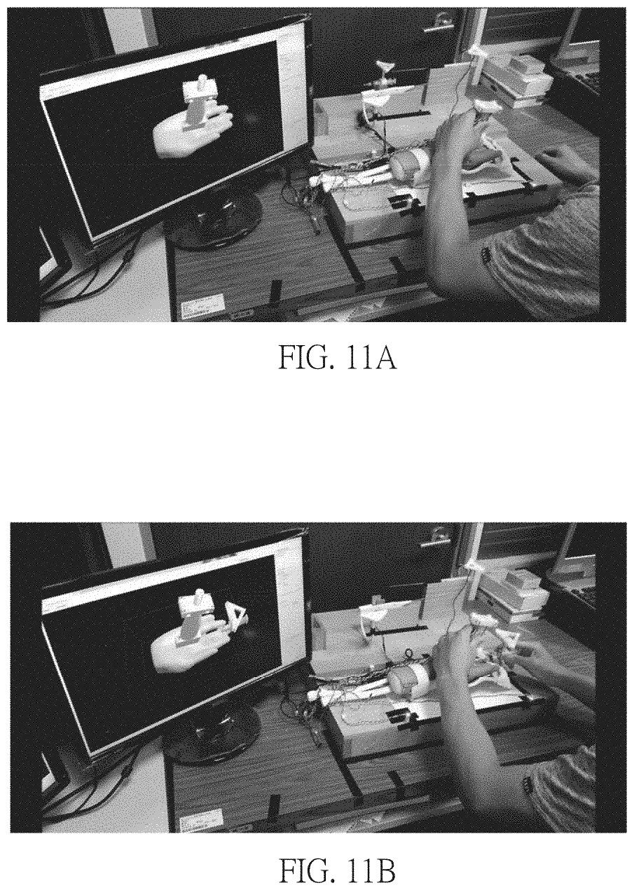

FIGS. 10A and 10B are schematic diagrams showing the hand phantom model and a calibration of ultrasound volume according to an embodiment of this disclosure. As shown in FIGS. 10A and 10B, the physical medical image 3-D model 14b and the artificial medical image 3-D model 14c are related to each other. Since the model of the hand phantom is constructed by the computed tomography image volume, the positional relationship between the computed tomography image volume and the ultrasonic volume can be directly used to create the relationship between the hand phantom and the ultrasound volume.

FIG. 10C is a schematic diagram showing a ultrasound volume and a collision detection according to an embodiment of this disclosure, and FIG. 10D is a schematic diagram showing an artificial ultrasound image according to an embodiment of this disclosure. As shown in FIGS. 10C and 10D, the training system is capable of simulating a real ultrasonic transducer (or probe) so as to produce a sliced image segment from the ultrasound volume. The simulated transducer (or probe) must depict the corresponding image segment regardless of the transducer (or probe) at any angle. In practice, the angle between the medical detection tool 21 and the ultrasonic body is first detected. Then, the collision detection of the segment surface is based on the width of the medical detection tool 21 and the ultrasonic volume, which can be used to find the corresponding value of the image segment being depicted. The generated image is shown in FIG. 10D. For example, if the artificial medical image data is a stereoscopic ultrasonic image, the stereoscopic ultrasonic image has a corresponding ultrasonic volume, and the content of the image segment to be drawn by the simulated transducer (or probe) can be generated according to the corresponding position of the stereoscopic ultrasonic image.

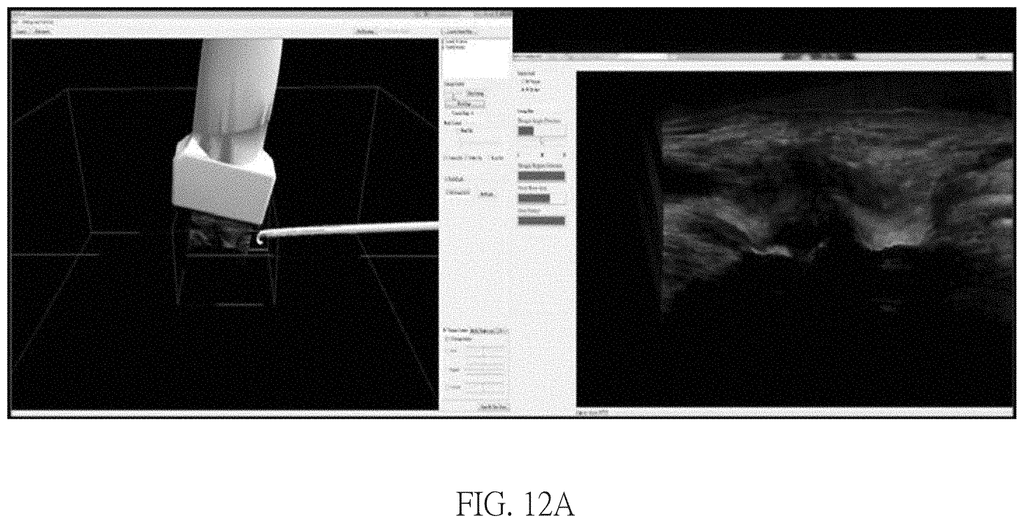

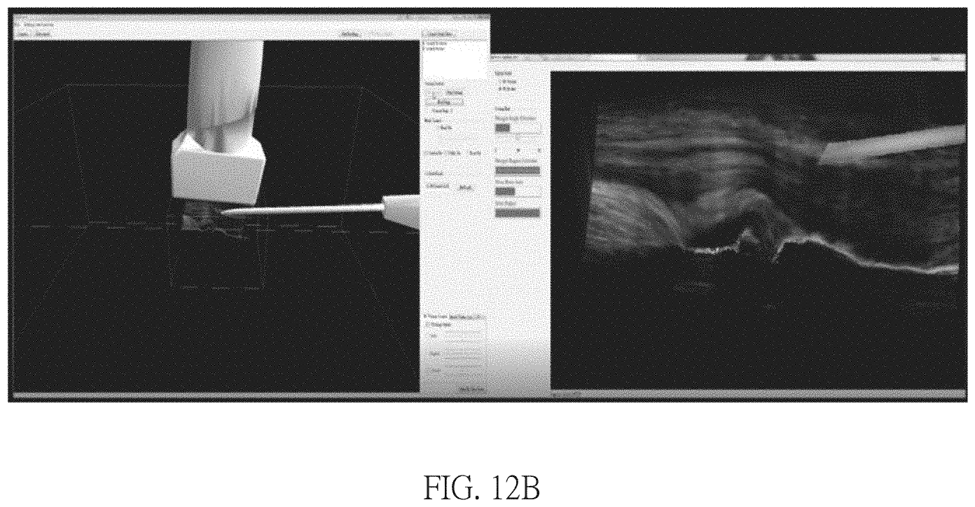

FIGS. 11A and 11B are schematic diagrams showing the operation training system according to an embodiment of this disclosure. As shown in FIGS. 11A and 11B, when the surgery trainee operates the medical instrument, the display device can show the image corresponding to the medical instrument in real-time. FIGS. 12A and 12B are schematic diagrams showing the images of the training system according to an embodiment of this disclosure. As shown in FIGS. 12A and 12B, when the surgery trainee operates the medical instrument, the display device can show the image corresponding to the medical instrument in real-time and the current artificial ultrasonic image in real-time.

In summary, the wearable image display device for surgery and the surgery information real-time display system of this disclosure can assist or train the user to operate the medical instrument. The training system of this disclosure can provide the trainee with a realistic surgical training situation, thereby effectively assisting the trainee to complete the surgical training.

In addition, the surgeon can practice with a simulated surgery on a fake body (e.g. a body phantom) in advance, and then before the actual surgery operation, the surgeon can review the practiced simulated surgery by the wearable image display device for surgery and the surgical information real-time display system for quickly reminding the key points of the operation or the points to be noted.

Moreover, the wearable image display device for surgery and the surgery information real-time display system of this disclosure can be applied to the actual operation process. For example, the medical images (e.g. ultrasonic images) can be transmitted to the wearable image display device for surgery (e.g. a smart glasses), so that the surgeon can view the displayed images without turning head to watch another display screen.

Although the disclosure has been described with reference to specific embodiments, this description is not meant to be construed in a limiting sense. Various modifications of the disclosed embodiments, as well as alternative embodiments, will be apparent to persons skilled in the art. It is, therefore, contemplated that the appended claims will cover all modifications that fall within the true scope of the disclosure.

What is claimed is:

1. A wearable image display device for surgery, comprising:
a display unit;
a wireless receiver receiving a medical image or a medical instrument information in real-time; and
a processing core coupled to the wireless receiver and the display unit for displaying the medical image or the medical instrument information on the display unit.

2. The device of claim 1, wherein the medical image is an artificial medical image of an artificial limb.

3. The device of claim 1, wherein the wearable image display device is a smart glasses or a head mounted display.

4. The device of claim 1, wherein the medical instrument information comprises a location information and an angle information.

5. The device of claim 1, wherein the wireless receiver wirelessly receives a surgery target information in real-time, and the processing core displays the medical image, the medical instrument information or the surgery target information on the display unit.

6. The device of claim 5, wherein the surgery target information comprises a location information and an angle information.

7. The device of claim 1, wherein the wireless receiver wirelessly receives a surgery guidance video in real-time, and the processing core displays the medical image, the medical instrument information or the surgery guidance video on the display unit.

8. A surgery information real-time display system, comprising:
the wearable image display device for surgery of claim 1; and
a server wirelessly connected with the wireless receiver for wirelessly transmitting the medical image and the medical instrument information in real-time.

9. The system of claim 8, wherein the server transmits the medical image and the medical instrument information through two network sockets, respectively.

10. The system of claim 8, further comprising:
an optical positioning device detecting a position of a medical instrument for generating a positioning signal, wherein the server generates the medical instrument information according to the positioning signal.

11. The system of claim 8, wherein the medical image is an artificial medical image of an artificial limb.

12. The system of claim 8, wherein the wearable image display device is a smart glasses or a head mounted display.

13. The system of claim 8, wherein the medical instrument information comprises a location information and an angle information.

14. The system of claim 8, wherein the wireless receiver wirelessly receives a surgery target information in real-time, and the processing core displays the medical image, the medical instrument information or the surgery target information on the display unit.

15. The system of claim 14, wherein the surgery target information comprises a location information and an angle information.

16. The system of claim 8, wherein the wireless receiver wirelessly receives a surgery guidance video in real-time, and the processing core displays the medical image, the medical instrument information or the surgery guidance video on the display unit.
